(19) United States
(12) Patent Application Publication
(10) Pub. No.: US 2008/0242657 A1
(43) Pub. Date: Oct. 2, 2008

(54) Treatment of Tremor with Histamine H3 Inverse Agonists or Hist Amine H3 Antagonists

(76) Inventors: Michael J. Marino, Souderton, PA (US); Guy R. Seabrook, Blue Bell, PA (US)

Correspondence Address: MERCK AND CO., INC, P O BOX 2000, RAHWAY, NJ, 07065-0907, US

(21) Appl. No.: 10/593,933

(22) PCT Filed: Mar. 22, 2005
(86) PCT No.: PCT/US05/09562

§ 371 Date: Sep. 21, 2006

Related U.S. Application Data
(60) Provisional application No. 60/556,803, filed on Mar. 26, 2004.
(60) Provisional application No. 60/561,188, filed on Apr. 9, 2004.
(60) Provisional application No. 60/629,099, filed on Nov. 18, 2004.

(51) Int. Cl. A61K 31/536; A61K 31/195; A61K 31/164; A61K 31/5513; A61K 31/5415; A61K 31/382; A61K 31/505; A61P 25/14; A61K 31/517; A61K 31/426; A61K 31/498; A61K 31/445; A61K 31/045; A61K 31/439

(52) U.S. Cl. 514/211.13; 514/567; 514/622; 514/213.01; 514/224.8; 514/437; 514/225.8; 514/249; 514/319; 514/221; 514/724; 514/286; 514/370; 514/269; 514/259.1; 514/290

(57) ABSTRACT

Histamine H3 inverse agonists or histamine H3 antagonists are useful, alone or in combination with a neuroleptic agent, for treating or preventing movement disorders, including tremor, such as essential tremor, and tremor associated with Parkinson's disease, cranofacial trauma, multiple sclerosis, stroke, dystonia, and neuropathic, toxic or drug induced tremor.

### BACKGROUND OF THE INVENTION

Diseases of the extrapyramidal motor systems cause either a loss of movement (akinesia) accompanied by an increase in muscle tone (rigidity) or abnormal involuntary movements (dyskinesias) often accompanied by a reduction in muscle tone. The akinetic-rigid syndrome called parkinsonism, and the dyskinesias represent opposite ends of the spectrum of movement disorders (for review see C. D. Marsden in Oxford Textbook of Medicine, 3rd Edition, Oxford University Press, 1996, vol. 3, pages 3998-4022).

Essential tremor is a disorder that affects 5-10 million persons in the United States. It is characterized primarily by an action and postural tremor most often affecting the arms, but it can also affect other body parts. Essential tremor is a progressive neurologic disorder and can cause substantial disability in some patients. Although there currently is no cure for essential tremor, pharmacologic and surgical treatments can provide some benefit. The clinical diagnosis of essential tremor is reviewed by Pahwa et al., Am. J. Medicine, 115, 134-142 (Aug. 1, 2003), and current treatments for essential tremor are reviewed by Lyons et al., Drug Safety, 26(7): 461-481 (2003).

Treatment of akinetic-rigid conditions such as parkinsonism typically involves the use of levodopa, anticholinergics or dopamine agonists. Levodopa is converted into dopamine in the brain by the enzyme dopa decarboxylase. However, this enzyme is also present in the gut wall, liver, kidney and cerebral capillaries, thus the peripheral formation of levodopa metabolites may give rise to side-effects such as nausea, vomiting, cardiac dysrhythmias and postural hypotension. This peripheral decarboxylation is largely prevented by the addition of a selective extracerebral decarboxylase inhibitor, such as carbidopa or benserazide, which themselves do not penetrate the brain. Levodopa combined with carbidopa (SINEMET™) or benserazide (MADOPAR™) is now the treatment of choice when levodopa is indicated. Even then, this combination therapy may be associated with side-effects such as dyskinesias and psychiatric disturbances.

Tremor, chorea, myoclonus, tics and dystonias, are treated with a variety of pharmacological agents. Thus, for example, tremor may be treated with benzodiazepines such as diazepam; chorea may be treated with diazepam, a phenothiazide or haloperidol, or tetrabenazine; tics may be controlled with neuroleptics such as haloperidol or pimozide; and dystonias tend to be treated with levodopa, benzodiazepines such as diazepam, anticholinergics such as benzhexyl, phenothiazines and other neuroleptics such as haloperidol, and tetrabenazine.

Treatment of psychotic disorders with neuroleptic agents, such as haloperidol may be at the expense of a number of side-effects, including extrapyramidal symptoms, acute dystonias, tardive dyskinesias, akathesia, tremor, tachycardia, drowsiness, confusion, postural hypotension, blurring of vision, precipitation of glaucoma, dry mouth, constipation, urinary hesitance and impaired sexual function. PCT Patent Publication WO 01/30346 discloses the use of histamine H3 agonists for the treatment of dyskinesia.

There exists a patient population in whom tremor is inadequately treated with existing neuroleptic therapy. Furthermore, some patients may be adversely affected by the side-effects of neuroleptic drugs. In view of the shortcomings of existing therapy, there is a need for new, safe and effective treatment for tremor and movement disorders.

### SUMMARY OF THE INVENTION

The present invention is directed to the use of a histamine H3 inverse agonist or antagonist, alone or in combination with a neuroleptic agent, for treating or preventing movement disorders, including tremor, such as essential tremor, and tremor associated with Parkinson's disease, cranofacial trauma, multiple sclerosis, stroke, dystonia, neuropathic induced tremor, toxic induced tremor, or drug induced tremor.

### DESCRIPTION OF THE INVENTION

The present invention is directed to the use of a histamine H3 inverse agonist or histamine H3 antagonist, or a pharmaceutically acceptable salt thereof, alone or in combination with a neuroleptic agent, for treating or preventing movement disorders, including tremor, such as essential tremor, and tremor associated with Parkinson's disease, cranofacial trauma, multiple sclerosis, stroke, dystonia, neuropathic induced tremor, toxic induced tremor, drug induced tremor, or tremor associated with dyskinesia, tardive diskinesia, drug-induced parkinsonism, postencephalitic parkinsonism, progressive supranuclear palsy, multiple system atrophy, corticobasal degeneration, parkinsonian-ALS dementia complex, basal ganglia calcification, akinesia, akinetic-rigid syndrome, bradykinesia, dystonia, medication-induced parkinsonian, Gilles de la Tourette syndrome, Huntingon disease, chorea, myoclonus, and tick disorder.

An embodiment of the present invention is directed to a method for treating, controlling, ameliorating or reducing the risk of a movement disorder in a patient in need thereof that comprises administering to the patient a therapeutically effective amount of a histamine H3 inverse agonist or histamine H3 antagonist, or a pharmaceutically acceptable salt thereof.

An embodiment of the present invention is directed to a method for treating, controlling, ameliorating or reducing the risk of a tremor, such as essential tremor, in a patient in need thereof that comprises administering to the patient a therapeutically effective amount of a histamine H3 inverse agonist or histamine H3 antagonist, or a pharmaceutically acceptable salt thereof

An embodiment of the present invention is directed to a method for treating, controlling, ameliorating or reducing the risk of a dyskinesia in a patient in need thereof who is non-responsive to neuroleptic agents or for whom neuroleptic agents are contraindicated, that comprises administering to the patient a therapeutically effective amount of a histamine H3 inverse agonist or histamine H3 antagonist, or a pharmaceutically acceptable salt thereof.

Although a histamine H3 inverse agonist or antagonist is useful alone for movement disorders, it will be appreciated that a combination of a conventional antiparkinsonian drug with a histamine H3 inverse agonist or antagonist may provide an enhanced effect in the treatment of tremor or akinetic-rigid disorders such as parkinsonism. Such a combination may enable a lower dose of the antiparkinsonian agent to be used without compromising the efficacy of the antiparkinsonian agent, thereby minimizing the risk of adverse side-effects.

An embodiment of the present invention is directed to a method for treating, controlling, ameliorating or reducing the risk of an akinetic-rigid disorder in a patient in need thereof, that comprises administering to the patient a therapeutically effective amount of a histamine H3 inverse agonist or histamine H3 antagonist, or a pharmaceutically acceptable salt thereof and an amount of an antiparkinsonian agent, such that together they give effective relief.

An embodiment of the present invention is directed to a method for treating, controlling, ameliorating or reducing the risk of a dyskinesia in a patient in need thereof, that comprises administering to the patient a therapeutically effective amount of a histamine H3 inverse agonist or histamine H3 antagonist, or a pharmaceutically acceptable salt thereof, and an amount of a neuroleptic agent, such that together they give effective relief.

It will be further appreciated that a combination of a conventional neuroleptic drug with a histamine H3 inverse agonist or antagonist or a pharmaceutically acceptable salt thereof may provide an enhanced effect in the treatment of dyskinesias. Such a combination may enable a lower dose of the neuroleptic agent to be used without compromising the efficacy of the neuroleptic agent, thereby minimising the risk of adverse side-effects. A yet further advantage of such a combination is that, due to the action of a histamine H3 inverse agonist or antagonist, adverse side-effects caused by the neuroleptic agent such as acute dystonias, dyskinesias, akathesia and tremor may be reduced or prevented.

The present invention also provides a method for the treatment or prevention of dyskinesias, which method comprises administration to a patient in need of such treatment of an amount of a histamine H3 inverse agonist or histamine H3 antagonist, or a pharmaceutically acceptable salt thereof and an amount of a neuroleptic agent, such that together they give effective relief.

In an embodiment of the present invention the histamine H3 inverse agonist is a selective histamine H3 inverse agonist. In an embodiment of the present invention the histamine H3 inverse agonist possesses a selectivity for the histamine H3 receptor relative to all other non-histamine G-protein coupled receptors of at least 5 fold as measured by the ratio of IC₅₀ for the histamine H3 receptor to the IC₅₀ for each of the other non-histamine G-protein coupled receptors. In another embodiment of the present invention the histamine H3 receptor inverse agonist possesses a selectivity for the histamine H3 receptor relative to all other non-histamine G-protein coupled receptors of at least 50 fold as measured by the ratio of IC₅₀ for the histamine H3 receptor to the IC₅₀ for each of the non-histamine G-protein coupled receptors. In another embodiment of the present invention the histamine H3 receptor inverse agonist possesses a selectivity for the histamine H3 receptor relative to all other non-histamine G-protein coupled receptors of at least 100 fold as measured by the ratio of IC₅₀ for the histamine H3 receptor to the IC₅₀ for each of the non-histamine G-protein coupled receptors. In another embodiment of the present invention the histamine 113 receptor inverse agonist possesses a selectivity for the histamine H3 receptor relative to other non-histamine G-protein coupled receptors of at least 200 fold as measured by the ratio of IC₅₀ for the histamine H3 receptor to the IC₅₀ for each of the other non-histamine G-protein coupled receptors. In an embodiment of the present invention the histamine H3 inverse agonist possesses a selectivity for the histamine H3 receptor relative to all other histamine receptors of at least 5 fold as measured by the ratio of IC₅₀ for the histamine H3 receptor to the IC₅₀ for each of the other histamine receptors. In another embodiment of the present invention the histamine H3 receptor inverse agonist possesses a selectivity for the histamine H3 receptor relative to all other histamine receptors of at least 50 fold as measured by the ratio of IC₅₀ for the histamine H3 receptor to the IC₅₀ for each of the other histamine receptors. In another embodiment of the present invention the histamine H3 receptor inverse agonist possesses a selectivity for the histamine H3 receptor relative to all other histamine receptors of at least 100 fold as measured by the ratio of IC₅₀ for the histamine H3 receptor to the IC₅₀ for each of the other histamine receptors. In another embodiment of the present invention the histamine H3 receptor inverse agonist possesses a selectivity for the histamine H3 receptor relative to the other histamine receptors of at least 200 fold as measured by the ratio of IC₅₀ for the histamine H3 receptor to the IC₅₀ for each of the other histamine receptors.

In an embodiment of the present invention the histamine H3 antagonist is a selective histamine H3 antagonist. In an embodiment of the present invention the histamine H3 antagonist possesses a selectivity for the histamine H3 receptor relative to all other non-histamine G-protein coupled receptors of at least 5 fold as measured by the ratio of IC₅₀ for the histamine H3 receptor to the IC₅₀ for each of the other non-histamine G-protein coupled receptors. In another embodiment of the present invention the histamine H3 receptor antagonist possesses a selectivity for the histamine H3 receptor relative to all other non-histamine G-protein coupled receptors of at least 50 fold as measured by the ratio of IC₅₀ for the histamine H3 receptor to the IC₅₀ for each of the non-histamine G-protein coupled receptors. In another embodiment of the present invention the histamine H3 receptor antagonist possesses a selectivity for the histamine H3 receptor relative to all other non-histamine G-protein coupled receptors of at least 100 fold as measured by the ratio of IC₅₀ for the histamine H3 receptor to the IC₅₀ for each of the non-histamine G-protein coupled receptors. In another embodiment of the present invention the histamine H3 receptor antagonist possesses a selectivity for the histamine H3 receptor relative to other non-histamine G-protein coupled receptors of at least 200 fold as measured by the ratio of IC₅₀ for the histamine H3 receptor to the IC₅₀ for each of the other non-histamine G-protein coupled receptors. In an embodiment of the present invention the histamine H3 antagonist possesses a selectivity for the histamine H3 receptor relative to all other histamine receptors of at least 5 fold as measured by the ratio of IC₅₀ for the histamine H3 receptor to the IC₅₀ for each of the other histamine receptors. In another embodiment of the present invention the histamine H3 receptor antagonist possesses a selectivity for the histamine H3 receptor relative to all other histamine receptors of at least 50 fold as measured by the ratio of IC₅₀ for the histamine H3 receptor to the IC₅₀ for each of the other histamine receptors. In another embodiment of the present invention the histamine H3 receptor antagonist possesses a selectivity for the histamine H3 receptor relative to all other histamine receptors of at least 100 fold as measured by the ratio of IC₅₀ for the histamine H3 receptor to the IC₅₀ for each of the other histamine receptors. In another embodiment of the present invention the histamine H3 receptor antagonist possesses a selectivity for the histamine H3 receptor relative to the other histamine receptors of at least 200 fold as measured by the ratio of IC₅₀ for the histamine H3 receptor to the IC₅₀ for each of the other histamine receptors.

In the present invention, a histamine H3 inverse agonist or antagonist may be employed as the free base or as a pharmaceutically acceptable salt thereof. Representative histamine H3 receptor ligands and pharmaceutically acceptable salts thereof are disclosed in e.g., U.S. Pat. Nos. 5,486,526; 5,652,258; 5,990,317; 6,008,240; 6,437,147 and PCT Patent Publications WO 96/40126; WO 96/38142; WO 01/300346; WO 01/068651; WO 01/068652; WO 02/015905; WO 03/004480; WO 03/024928; WO 03/066604; WO 03/0236259; and may be prepared by methods described therein. Representative histamine H3 inverse agonists include: 4-{(1R,2R)-trans-2-[O-(2-cyclohexylethyl)carboxamido]-cyclopropyl) imidazole, 4-{(1R,2R)-trans-2-[O-2-cyclohexylmethyl)carboxamido]cyclopropyl)-imidazole; 3-(1H-imidazol-4 yl)propyl-di(p-fluorophenyl)-methyl ether; and 2-(1-cyclopentylpiperidine-4-yloxy)-5-(4-cyanophenyl)pyrimidine.

The identification of a compound as a histamine H3 inverse agonist or a histamine H3 antagonist may be readily determined without undue experimentation by methodology well known in the art. The efficacy of a histamine H3 inverse agonist or a histamine H3 antagonist in treating tremor may be readily determined without undue experimentation by methodology well known in the art, for example the harmaline-induced tremor model in rats, Sinton, et al., Pflugers Archive Eur. J. Phys., 414(1) 31-36 (1989). In this model, a histamine H3 inverse agonist exhibited a dose dependent ability to decrease harmaline-induced tremor. In particular, at a dose of 1 mg/kg the histamine H3 inverse agonist 2-(1-cyclopentylpiperidine-4-yloxy)-5-(4-cyanophenyl)pyrimidine exhibited an 8.4% reversal of harmaline-induced tremor; at a dose of 3 mg/kg the histamine H3 inverse agonist exhibited an 63.3% reversal of harmaline-induced tremor; and at a dose of 10 mg/kg the histamine H3 inverse agonist exhibited an 84.4% reversal of harmaline-induced tremor. The ability of the histamine H3 inverse agonist or histamine H3 antagonist to be used in the present invention to treat tremor or movement disorders may be determined by these methods.

2-(1-Cyclopentylpiperidine-4-yloxy)-5-(4-cyanophenyl) pyrimidine: To a DMF solution (10 mL) of 2-chrolo-5-bromo-pyrimidine (300 mg, 1.56 mmol), 1-tert-butoxy carbonyl-4-hydroxy-piperidine (408 mg, 2.03 mmol) and cesium carbonate were added. The reaction mixture was stirred for 14 hours at room temperature. Water was added to a reaction mixture and the reaction mixture was extracted with ethyl acetate. The organic layer was washed with brine, dried over anhydrous sodium sulfate and concentrated in vacuo. The residue was purified by silica gel column chromatography (C-300, hexane:ethyl acetate=10:1) to afford 2-(1-tert-butoxycarbonylpiperidine-4-yloxy)-5-bromopyrimidine. To a 2-(1-tert-butoxycarbonylpiperidine-4-yloxy)-5-(4-cyanophenyl) pyrimidine (149 mg, 0.42 mmol), 2-dimethoxyethane (2.0 mL) and 2N sodium carbonate (0.7 mL) were added, and then 4-cyano-boric acid (75.2 mg, 0.51 mmol) and tetrakis(triphenyl phosphine)palladium(0) (10 mg, 0.0087 mmol) were added. The reaction mixture was stirred at 90 degree for 30 hours under N2 atmosphere. After the reaction mixture was cooled to room temperature, and extracted with ethyl acetate. The organic layer was washed with brine, dried over anhydrous sodium sulfate, and concentrated in vacuo. The residue was purified by silica gel column chromatography (C-300, hexane:ethyl acetate=3:1) to afford 2-(1-tert-butoxycarbonylpiperidine-4-yloxy)-5-(4-cyanophenyl)pyrimidine. To a methylene chloride solution of 2-(1-tert-butoxycarbonylpiperidine-4-yloxy)-5-(4-cyanophenyl) pyrimidine (122 mg, 0.32 mmol) was added trifluoroacetic acid (1.5 ml) and the reaction mixture was stirred at room temperature for 2.5 hours. The reaction mixture was concentrated in vacuo and the residue was extracted with chloroform. The organic layer was sodium hydrogen carbonate, brine, dried over anhydrous sulfate and concentrated in vacuo to afford 2-(piperidine-4-yloxy)-5-(4-cyanophenyl)-pyrimidine. To a methanol solution (3.0 mL) of 2-(pyperidine-4-yloxy)-5-(4-cyanophenyl)pyrimidine (46 mg, 0.16 mmol), cyclopentanone and 0.3N zinc chloride-sodium cyanoborate solution (0.55 mL) were added and the reaction mixture was stirred at room temperature for 14 hours. The reaction mixture was concentrated in vacuo and the residue was extracted with chloroform. The organic layer was washed with brine, dried over anhydrous sodium sulfate and concentrated in vacuo. The residue obtained was purified by silica gel column chromatography (eluted with chloroform:methanol=10:1) to afford 2-(1-cyclopentylpiperidine-4-yloxy)-5-(4-cyanophenyl) pyrimidine. ¹H NMR (300 MHz, CDCl₃, δ ppm): 1.38-1.78 (6H, m), 1.82-2.04 (4H, m), 2.08-2.21 (2H, m), 2.32-2.63(3H, m), 2.74-2.96(2H, m), 5.07-5.18(1H, m), 7.62(2H, d, J=8.6 Hz), 7.78(1H, d, J=8.6 Hz), 8.73(2H, s), Mass(ESI): 349(M+H).

As used herein, the term “movement disorders” includes akinesias and akinetic-rigid syndromes, dyskinesias and medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor). Examples of “akinetic-rigid syndromes” include Parkinson's disease, drug-induced parkinsonisin, postencephalitic parkinsonism, progressive supranuclear palsy, multiple system atrophy, corticobasal degeneration, parkinsonism-ALS dementia complex and basal ganglia calcification. Examples of “dyskinesias” include chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia). Another “movement disorder” which may be treated according to the present invention is Gilles de la Tourette's syndrome, and the symptoms thereof.

As used herein, the term “treatment” refers both to the treatment and to the prevention or prophylactic therapy of the aforementioned conditions. The term “therapeutically effective amount” shall mean that amount of a drug or pharmaceutical agent that will elicit the biological or medical response of a tissue, system, animal or human that is being sought by a researcher or clinician.

Accordingly, the present invention includes within its scope the use of a histamine H3 inverse agonist or antagonist, alone or in combination with other agents, for the subject indications in a warm-blooded animal. For the purposes of this disclosure, a warm-blooded animal is a member of the animal kingdom which includes but is not limited to mammals and birds. The preferred mammal for purposes of this invention is human.

The subject treated in the present methods is generally a mammal, preferably a human, male or female. In the present invention, it is preferred that the subject mammal is a human. Although the present invention is applicable both old and young people, in certain aspects such as cognition enhancement it would find greater application in elderly people. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof. The terms “administration of” and or “administering a” compound should be understood to mean providing a compound of the invention or a prodrug of a compound of the invention to the individual in need of treatment.

This particular application of a histamine H3 inverse agonist or antagonist provides unexpected benefit relative to the administration of other agents for the subject indications. For example, a histamine H3 inverse agonist or antagonist may exhibit a rapid onset of action and a reduced side-effect profile relative to conventional agents used for the treatment of extrapyramidal movement disorders and other types of movement disorders (e.g. idiopathic Parkinson's disease, secondary Parkinson's disease, Huntingdon's disease, dystonia, chorea, tics, myoclonus and athetosis).

For use in medicine, the salts of the compounds employed in this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free base with a suitable organic or inorganic acid. Representative salts include the following: Acetate, Benzenesulfonate, Benzoate, Bicarbonate, Bisulfate, Bitartrate, Borate, Bromide, Calcium, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Subacetate, Succinate, Sulfate, Sulfonate, Tannate, Tartrate, Teoclate, Tosylate, Triethiodide and Valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds employed in the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

A histamine H3 inverse agonist or antagonist may be used alone or in combination with other neuroleptic agents or with other compounds which are known to be beneficial in the subject indications. A histamine H3 inverse agonist or antagonist and the other agent may be co-administered, either in concomitant therapy or in a fixed combination. For example, a histamine H3 inverse agonist or antagonist may be administered in conjunction with other compounds which are known in the art for the subject indications. It will be appreciated that when using a combination of the present invention, a histamine H3 inverse agonist or antagonist and the antiparkinsonian or neuroleptic agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the antiparkinsonian or neuroleptic agent may be administered as a tablet and then, within a reasonable period of time, a histamine H3 inverse agonist or antagonist may be administered either as an oral dosage form such as a tablet or a fast-dissolving oral dosage form. By a “fast-dissolving oral formulation” is meant, an oral delivery form which when placed on the tongue of a patient, dissolves within about 10 seconds.

In accordance with the present invention, a histamine H3 inverse agonist or antagonist is useful alone or in combination with other antipsychotic agents for treating, controlling, ameliorating or reducing the risk of a movement disorder.

Suitable antiparkinsonian agents of use in combination with a histamine H3 inverse agonist or antagonist include levodopa (with or without a selective extracerebral decarboxylase inhibitor such as carbidopa or benserazide), anticholinergics such as biperiden (optionally as its hydrochloride or lactate salt) and trihexyphenidyl (benzhexyl)hydrochloride, and dopamine agonists such as alentemol, bromocriptine, fenoldopam, lisuride, naxagolide, pergolide, pramipexole, entacapone, an anticholinergic, a COMT inhibitor, an A2a adenosine receptor antagonist, a cholinergic agonist, a dopamine agonist, a butyrophenone neuroleptic agent, a diphenylbutylpiperidine neuroleptic agent, a heterocyclic dibenzazepine neuroleptic agent, a indolone neuroleptic agent, a phenothiazine neuroleptic agent, a thioxanthene neuroleptic agent, an NMDA receptor antagonist, a metabotropic glutarnate receptor potentiator and a metabotropic glutamate receptor agonist. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

A histamine H3 inverse agonist or antagonist or a pharmaceutically acceptable salt thereof, may be administered in combination with a compound selected from the group consisting of: acetophenazine, alentemol, benzhexyl, bromocriptine, biperiden, chlorpromazine, chlorprothixene, clonazepam, clozapine, diazepam, ethanol, fenoldopam, fluphenazine, gabapentin, haloperidol, levodopa, levodopa with benserazide, levodopa with carbidopa, lisuride, loxapine, mesoridazine, molindolone, naxagolide, olanzapine, pergolide, perphenazine, pimozide, pramipexole, primidone, propranolol, risperidone, sulpiride, tetrabenazine, trihexyphenidyl, thioridazine, thiothixene and trifluoperazine, or is administered following surgery, such as surgical intervention by thalamic lesion.

Suitable neuroleptic agents of use in combination with a histamine H3 inverse agonist or antagonist or a pharmaceutically acceptable salt thereof include the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with a histamine H3 inverse agonist or antagonist may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form.

The present invention includes within its scope a pharmaceutical composition for the subject indications comprising, as an active ingredient, a histamine H3 inverse agonist or antagonist in association with a pharmaceutical carrier or diluent. Optionally, the active ingredient of the pharmaceutical compositions can comprise another agent in addition to a histamine H3 inverse agonist or antagonist to minimize the side effects or with other pharmaceutically active materials wherein the combination enhances efficacy and minimizes side effects.

The present invention is further directed to a method for the manufacture of a medicament for the subject indications in humans comprising combining a compound that is a histamine H3 inverse agonist or antagonist with a pharmaceutical carrier or diluent.

It will be known to those skilled in the art that there are numerous compounds now being used for movement disorders. Combinations of these therapeutic agents some of which have also been mentioned herein with a histamine H3 inverse agonist or antagonist will bring additional, complementary, and often synergistic properties to enhance the desirable properties of these various therapeutic agents. In these combinations, a histamine H3 inverse agonist or antagonist and the therapeutic agents may be independently present in dose ranges from one one-hundredth to one times the dose levels which are effective when these compounds and secretagogues are used singly.

To illustrate these combinations, a histamine H3 inverse agonist or antagonist effective clinically at a given daily dose range may be effectively combined, at levels which are equal or less than the daily dose range, with such compounds at the indicated per day dose range. Typically, the individual daily dosages for these combinations may range from about one-fifth of the minimally recommended clinical dosages to the maximum recommended levels for the entities when they are given singly. It will be readily apparent to one skilled in the art that a histamine H3 inverse agonist or antagonist may be employed with other agents for the purposes of the present invention.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. A histamine H3 inverse agonist or antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the unit dosage form is a capsule, it may contain, in addition to materials of the above type, a liquid carrier such as fatty oil. Various other materials may be present as coatings or to otherwise modify the physical form of the dosage unit. Tablets and pills can additionally be prepared with enteric coatings and tablets may be coated with shellac, sugar or both.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art. Compositions for nasal or sublingual administration are also prepared with standard excipients well known in the art.

It will be appreciated that the amount of a histamine H3 inverse agonist or antagonist will vary not only with the compositions selected but also with the route of administration, the nature of the condition being treated, and the age and condition of the patient, and will ultimately be at the discretion of the patient's physician or pharmacist.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg of body weight daily are administered to the patient, e.g., humans and elderly humans. The dosage range will generally be about 0.5 mg to 1.0 g. per patient per day which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages for administration include 10 mg, 30 mg and 60 mg.

Pharmaceutical compositions of the present invention may be provided in a solid dosage formulation preferably comprising about 0.5 mg to 500 mg active ingredient, more preferably comprising about 1 mg to 250 mg active ingredient. The pharmaceutical composition is preferably provided in a solid dosage formulation comprising about 1 mg, 5 mg, 10 mg, 25 mg, 50 mg, 100 mg, 200 mg or 250 mg active ingredient.

A minimum dosage level for the antiparkinsonian agent will vary depending upon the choice of agent, but is typically about 0.05 mg per day for the most potent compounds or about 20 mg per day for less potent compounds. A maximum dosage level for the antipsychotic agent is typically 30 mg per day for the most potent compounds or 500 mg per day for less potent compounds. The compounds are administered one to three times daily, preferably once or twice a day, and especially once a day.

A minimum dosage level for the neuroleptic agent will vary depending upon the choice of agent, but is typically about 0.5 mg per day for the most potent compounds or about 20 mg per day for less potent compounds. A maximum dosage level for the neuroleptic agent is typically 30 mg per day for the most potent compounds or 200 mg per day for less potent compounds. The compounds are administered one to three times daily, preferably once or twice a day, and especially once a day.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-15. (canceled)

16. A method for treating, controlling, ameliorating or reducing the risk of essential tremor in a patient in need thereof that comprises administering to the patient a therapeutically effective amount of a histamine H3 inverse agonist or a pharmaceutically acceptable salt thereof.

17. The method of claim 16 wherein the histamine H3 inverse agonist is a selective inverse agonist of the histamine H3 receptor.

18. The method of claim 16 wherein the histamine H3 inverse agonist, or a pharmaceutically acceptable salt thereof, is administered in combination with an agent selected from the group consisting of: levodopa, levodopa with a selective extracerebral decarboxylase inhibitor, carbidopa, entacapone, an anticholinergic, a COMT inhibitor, an A2a adenosine receptor antagonist, a cholinergic agonist, a dopamine agonist, a butyrophenone neuroleptic agent, a diphenylbutylpiperidine neuroleptic agent, a heterocyclic dibenzazepine neuroleptic agent, a indolone neuroleptic agent, a phenothiazine neuroleptic agent, a thioxanthene neuroleptic agent, an NMDA receptor antagonist, a metabotropic glutamate receptor potentiator and a metabotropic glutamate receptor agonist.

19. The method of claim 16 wherein the histamine H3 inverse agonist, or a pharmaceutically acceptable salt thereof, is administered in combination with a compound selected from the group consisting of: acetophenazine, alentemol, benzhexyl, bromocriptine, biperiden, chlorpromazine, chlorprothixene, clonazepam, clozapine, diazepam, ethanol, fenoldopam, fluphenazine, gabapentin, haloperidol, levodopa, levodopa with benserazide, levodopa with carbidopa, lisuride, loxapine, mesoridazine, molindolone, naxagolide, olanzapine, pergolide, perphenazine, pimozide, pramipexole, primidone, propranolol, risperidone, sulpiride, tetrabenazine, trihexyphenidyl, thioridazine, thiothixene and trifluoperazine, or is administered following surgery.

20. A method for treating, controlling, ameliorating or reducing the risk of essential tremor in a patient in need thereof that comprises administering to the patient a therapeutically effective amount of a histamine H3 antagonist or a pharmaceutically acceptable salt thereof.

21. The method of claim 20 wherein the histamine H3 antagonist is a selective antagonist of the histamine H3 receptor.

22. The method of claim 20 wherein the histamine H3 antagonist, or a pharmaceutically acceptable salt thereof, is administered in combination with an agent selected from the group consisting of: levodopa, levodopa with a selective extracerebral decarboxylase inhibitor, carbidopa, entacapone, an anticholinergic, a COMT inhibitor, an A2a adenosine receptor antagonist, a cholinergic agonist, a dopamine agonist, a butyrophenone neuroleptic agent, a diphenylbutylpiperidine neuroleptic agent, a heterocyclic dibenzazepine neuroleptic agent, a indolone neuroleptic agent, a phenothiazine neuroleptic agent, a thioxanthene neuroleptic agent, an NMDA receptor antagonist, a metabotropic glutamate receptor potentiator and a metabotropic glutamate receptor agonist.

23. The method of claim 20 wherein the histamine H3 antagonist, or a pharmaceutically acceptable salt thereof, is administered in combination with a compound selected from the group consisting of: acetophenazine, alentemol, benzhexyl, bromocriptine, biperiden, chlorpromazine, chlorprothixene, clonazepam, clozapine, diazepam, ethanol, fenoldopam, fluphenazine, gabapentin, haloperidol, levodopa, levodopa with benserazide, levodopa with carbidopa, lisuride, loxapine, mesoridazine, molindolone, naxagolide, olanzapine, pergolide, perphenazine, pimozide, pramipexole, primidone, propranolol, risperidone, sulpiride, tetrabenazine, trihexyphenidyl, thioridazine, thiothixene and trifluoperazine, or is administered following surgery.

24. A method for treating, controlling, ameliorating or reducing the risk of an akinetic-rigid disorder in a patient in need thereof, that comprises administering to the patient a therapeutically effective amount of a histamine H3 inverse agonist or a histamine H3 antagonist, or a pharmaceutically acceptable salt thereof, and an amount of an antiparkinsonian agent, such that together they give effective relief.

25. The method of claim 24 wherein the histamine H3 inverse agonist is a selective inverse agonist of the histamine H3 receptor.

26. The method of claim 24 wherein the histamine H3 antagonist is a selective antagonist of the histamine H3 receptor.

27. The method of claim 24 wherein the histamine H3 inverse agonist or the histamine H3 antagonist, or a pharmaceutically acceptable salt thereof, is administered in combination with an agent selected from the group consisting of: levodopa, levodopa with a selective extracerebral decarboxylase inhibitor, carbidopa, entacapone, an anticholinergic, a COMT inhibitor, an A2a adenosine receptor antagonist, a cholinergic agonist, a dopamine agonist, a butyrophenone neuroleptic agent, a diphenylbutylpiperidine neuroleptic agent, a heterocyclic dibenzazepine neuroleptic agent, a indolone neuroleptic agent, a phenothiazine neuroleptic agent, a thioxanthene neuroleptic agent, an NMDA receptor antagonist, a metabotropic glutamate receptor potentiator and a metabotropic glutamate receptor agonist.

28. The method of claim 24 wherein the histamine H3 inverse agonist or the histamine H3 antagonist, or a pharmaceutically acceptable salt thereof, is administered in combination with a compound selected from the group consisting of: acetophenazine, alentemol, benzhexyl, bromocriptine, biperiden, chlorpromazine, chlorprothixene, clonazepam, clozapine, diazepam, ethanol, fenoldopam, fluphenazine, gabapentin, haloperidol, levodopa, levodopa with benserazide, levodopa with carbidopa, lisuride, loxapine, mesoridazine, molindolone, naxagolide, olanzapine, pergolide, perphenazine, pimozide, pramipexole, primidone, propranolol, risperidone, sulpiride, tetrabenazine, trihexyphenidyl, thioridazine, thiothixene and trifluoperazine, or is administered following surgery.
